(19) United States
(12) Patent Application Publication
(10) Pub. No.: US 2009/0247642 A1
(43) Pub. Date: Oct. 1, 2009

(54) SYNTHESIS AND PREPARATIONS OF METOPROLOL AND ITS SALTS

(76) Inventors: Carmen Arnalot Aguilar, Girona (ES); Jordi Bosch I Llado, Girona (ES)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 12/158,950

(22) PCT Filed: Dec. 22, 2006
(86) PCT No.: PCT/IB2006/004081

§ 371 Date: Jan. 8, 2009

Related U.S. Application Data
(60) Provisional application No. 60/752,949, filed on Dec. 23, 2005.

(51) Int. Cl. A61K 31/138; C07C 217/64; C07C 213/00

(52) U.S. Cl. 514/652; 564/348

(57) ABSTRACT

The invention relates to an improved process for preparing metoprolol and its salts.

### CROSS REFERENCE TO RELATED APPLICATIONS

This application claims priority to U.S. Provisional Application No. 60/752,949, filed Dec. 23, 2005, which is expressly incorporated herein by reference in its entirety.

### BACKGROUND OF THE INVENTION

1. Field of the Invention

The invention relates to an improved process for preparing metoprolol and its salts.

2. Discussion of the Related Art

Metoprolol succinate is a commercially marketed pharmaceutically active substance known to be useful for the treatment of hypertension, in the long-term treatment of angina pectoris and for the treatment of stable, symptomatic heart failure of ischemic, hypertensive and cardiomyopathic origin. Metoprolol succinate has an empirical formula of C15H25NO3.½C4H6O4 and a molecular weight of 652.8. Metoprolol succinate is the international common accepted name for (±) 1-(isopropylamino)-3-[p-(2-methoxyethyl)phenoxy]-2-propanol succinate (2:1) (salt), which is represented in Formula I.

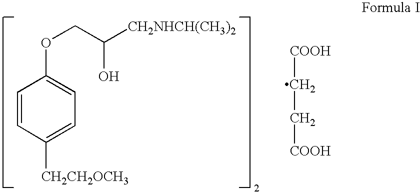

Metoprolol and its pharmaceutically acceptable salts are described in U.S. Pat. No. 3,998,790.

There are various known mechanisms for producing metoprolol and its salts. For example, EP 0 050 885 describes reaction of the corresponding epoxide, obtained from 4-(2-methoxyethyl)phenol and epichlorohydrin, with isopropylamine and a Lewis acid to produce metoprolol.

U.S. Pat. No. 5,082,969 describes a process, as shown in Scheme 1, for preparing metoprolol involving the treatment of 4-(2-methoxyethyl)phenol with aqueous sodium hydroxide and the epichlorhydrin at 0°-25° C. for 15-20 hours, to give the corresponding epoxide. The resulting epoxide is then reacted with a large excess of aqueous isopropylamine at 30° C. to yield metoprolol.

U.S. Pat. No. 6,252,113 describes a process where the epoxide obtained from the reaction of 4-(2-methoxyethyl)phenol and epichlorohydrin in aqueous alkaline conditions at 50-70° C. is distilled under high vacuum to improve quality. The epoxide is then treated with isopropylamine in isopropyl alcohol at reflux temperature, or in absence of isopropyl alcohol at 70±10° C., under pressure of 275-315 kPa to yield metoprolol.

Metoprolol succinate is first specifically mentioned in U.S. Pat. No. 5,081,154, although no examples for its preparation are provided. U.S. Patent Application Publication No. 20050107635 describes, for the first time, experimental conditions to convert metoprolol base into metoprolol succinate. The salt is made in an acetone medium, and the crude metoprolol salt is recrystallized from methanol to obtain purified metoprolol succinate.

In most of the known processes, including those described above, drastic temperature and pressure conditions, expensive raw materials, a catalyst or large amounts of noxious organic solvents are used. There is, therefore, a need for improved processes for preparing metoprolol and/or its salts.

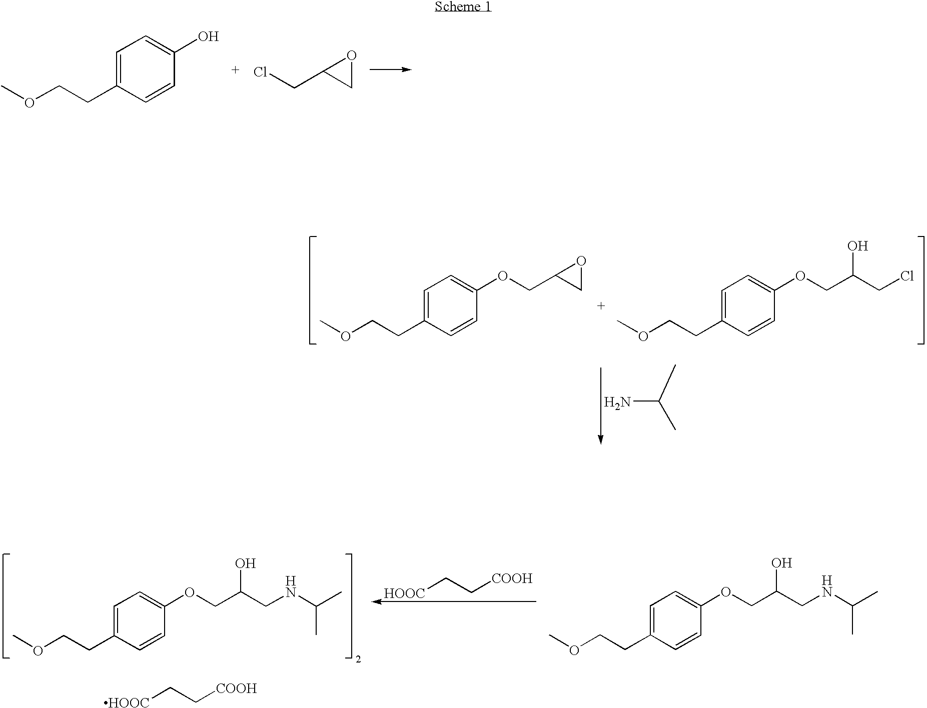

### SUMMARY OF THE INVENTION

The invention relates to an improved process for preparing metoprolol and its salts.

One aspect of the invention includes a process involving the reaction of 4-(2-methoxyethyl)phenol with (R,S)-epichlorhydrin in aqueous alkaline conditions at a temperature of approximately 35±2° C., characterized in that the base is added in two portions. The resulting epoxide intermediate obtained is then reacted with isopropylamine at approximately 50-55° C. in the absence of a solvent to yield metoprolol base. The resulting metoprolol base can then optionally be converted to one of its pharmaceutically acceptable salts including, for example, its succinate salt.

A further aspect of the invention includes the reaction between the epoxide and the isopropylamine, as described above, being performed in the absence of solvents and at atmospheric pressure.

A further aspect of the invention includes the conversion of metoprolol base into a metoprolol salt (e.g., the succinate salt) being performed in an alcoholic solvent without the need to perform additional purification and/or crystallization steps to produce a metoprolol salt (e.g., the succinate salt) of suitable pharmaceutical quality.

A further aspect of the invention includes the addition of a base, as described above and which is preferably potassium hydroxide, that is added in two portions and where addition of the second portion of the base after some hours of reaction helps to complete the reaction and gives better results than the addition of all the required base at the beginning of the reaction.

A further aspect of the invention includes a process for preparing metoprolol succinate from metoprolol base in isopropanol, which is advantageous compared to previously known processes employing more noxious solvents (e.g., methanol).

A further aspect of the invention includes a process for drying metoprolol succinate in which the temperature used for drying metoprolol succinate is preferably between approximately 85° C. and approximately 100° C. and more preferably between approximately 90° C. and approximately 95° C., whereby drying at these temperatures the resulting metoprolol succinate has a maximum loss on drying of approximately 0.2% of its weight, and whereby drying the product at higher temperatures such as 110° C. or above results in product degradation.

A further aspect of the invention includes providing metoprolol succinate of defined particle size, including a plurality of metoprolol succinate particles.

A further aspect of the invention includes characterizing metoprolol succinate by its X-ray powder diffractogram spectrum.

Additional advantages and features of the invention will become apparent from the detailed description which follows.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates the X-ray powder diffractogram of metoprolol succinate where the horizontal axis presents 2θ and the vertical axis corresponds to the peak intensity.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition, and as will be appreciated by one of skill in the art, the invention may be embodied as a method, system or process.

The invention relates to an improved process for preparing metoprolol and its salts.

One aspect of the invention includes a first step (Step A) in which 4-(2-methoxyethyl)phenol is reacted with (R,S)-epichlorhydrin in an aqueous alkaline solution at approximately 35±2° C. and characterized in that the base is added in two portions, where the resulting water-epichlorhydrin mixture is distilled under vacuum and where a distillation residue is obtained.

Another aspect of the invention includes a second step (Step B) in which the distillation residue obtained in Step A is reacted with isopropylamine while keeping the temperature below approximately 15° C. The suspension thus obtained is then heated at reflux temperature (approximately 50° C. to approximately 55° C.) and kept at reflux for approximately 3 hours. Excess isopropylamine is removed by distillation at atmospheric pressure while ensuring that the reaction temperature does not exceed approximately 70±3° C. The resulting metoprolol base obtained is then extracted as a toluenic solution and washed by conventional methods. Next, the toluenic solution of metoprolol base is distilled under vacuum while ensuring that the temperature does not exceed approximately 80° C. The resulting residue is then cooled to approximately 60±5° C., isopropanol is added, and the mixture is cooled to room temperature.

Another aspect of the invention includes a third step (Step C) in which a solution of succinic acid in isopropanol is prepared and heated to approximately 55° C. to approximately 65° C. Then, the solution is filtered and added to the isopropanolic solution of metoprolol base obtained in Step B, which has been previously filtered and heated to approximately 55° C. to approximately 65° C. The mixture is then cooled to approximately 20° C. to approximately 25° C., stirred for approximately 2 hours, filtered and washed with filtered isopropanol. Additional filtered isopropanol is added to the crude product obtained and, after stirring the mixture for approximately 1 hour, the solution is filtered, washed and dried to yield metoprolol succinate.

Another aspect of the invention includes an improved process for preparing metoprolol succinate from metoprolol base which includes:

i. preparing a solution of succinic acid in isopropanol and heating this solution to approximately 55° C. to approximately 65° C.;

ii. adding the solution obtained in the previous step to a isopropanolic solution of metoprolol base, which has been previously heated to approximately 55° C. to approximately 65° C.;

iii. maintaining the solution at approximately 55° C. to approximately 65° C. for about approximately 30 additional minutes;

iv. cooling the solution to approximately 20° C. to approximately 25° C. over a period of approximately 2 hours;

v. stirring the solution for approximately 2 hours at approximately 20° C. to approximately 25° C.;

vi. filtering, isolating and washing the resulting product (i.e., metoprolol succinate) with isopropanol;

vii. drying the resulting product (i.e., metoprolol succinate) under vacuum at a temperature of approximately 85° C. to approximately 95° C.; and

viii. optionally milling and sieving the resulting product (i.e., metoprolol succinate).

Another aspect of the invention includes a drying process for use in the preparation of metoprolol succinate that includes drying metoprolol succinate at a temperature of approximately 85° C. to approximately 95° C., and preferably at a temperature of approximately 90° C. to approximately 95° C.

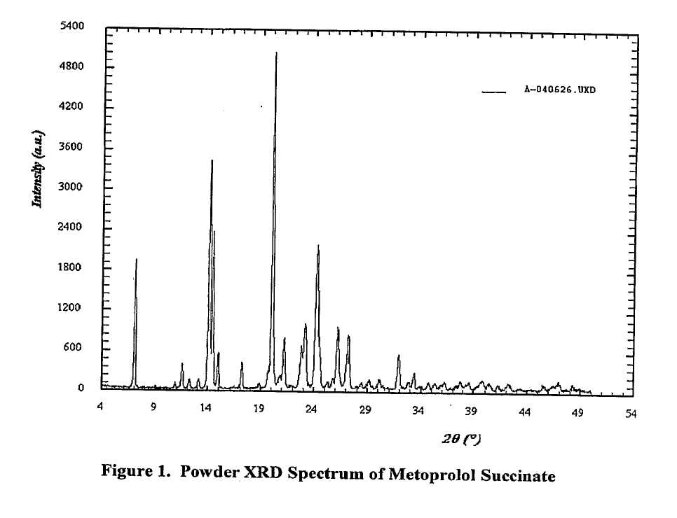

Another aspect of the invention includes metoprolol succinate characterized by powder X-ray spectrum. As depicted in FIG. 1, metoprolol succinate is characterized by its X-Ray powder diffraction pattern (2θ) (±0.2°) (XRD) as having peaks at approximately 7.1, 11.5, 12.2, 13.1, 14.1, 14.4, 14.9, 17.2, 20.1, 21.2, 22.8, 23.1, 24.3, 24.6, 25.8, 26.2, 27.2, 30.1, 31.9, 33.4°.

Another aspect of the invention includes a process for preparing metoprolol succinate characterized by the powder X-ray spectrum depicted in FIG. 1.

Another aspect of the invention includes a powder composition including metoprolol succinate, wherein the metoprolol succinate has a particle size distribution in which approximately 10% of the particles have a diameter below approximately 5 μm, approximately 50% of the particles have a diameter below about approximately 20 μm and approximately 90% of the particles have a diameter below approximately 55 μm.

Another aspect of the invention includes a powder composition prepared by milling a metoprolol succinate feedstock having a mean particle size of approximately 25 μm.

Another aspect of the invention includes a dosage form including metoprolol succinate, wherein the metoprolol succinate has a particle size distribution in which approximately 10% of the particles have a diameter below approximately 5 μm, approximately 50% of the particles have a diameter below about approximately 20 μm and approximately 90% of the particles have a diameter below approximately 55 μm.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

### SPECIFIC EXAMPLES

The following examples are for illustrative purposes only and are not intended, nor should they be interpreted to, limit the scope of the invention.

General Experimental Conditions:

HPLC Method

The chromatographic separation was carried out in a Sperspher RP-select B (brand L7), 4 μm, 125×4.0 mm I.D. column at 30° C.

The mobile phase was prepared by mixing 400 mL of acetonitrile with 600 mL of sodium dodecyl sulphate solution, which was prepared by dissolving 1.3 g of sodium dodecyl sulfate in 1000 ml of aqueous phosphoric acid 0.1%. The mobile phase was mixed and filtered through 0.22 μm nylon membrane under vacuum.

The chromatograph was equipped with a 223 nm detector, and the flow rate was 0.9 mL per minute. Test samples (10 μL) were prepared by dissolving the appropriate amount of sample in the mobile phase in order to obtain 1 mg per mL of mobile phase.

### Particle Size Analysis:

The particle size for metoprolol succinate was measured using a Malvern Mastersizer S particle size analyzer with an MS1 Small Volume Recirculating unit attached. A 300RF mm lens and a beam length of 2.4 mm were used.

Samples for analysis were prepared by dispersing a weighed amount of metoprolol succinate (approximately 0.1 g) in 20 mL of toluene. The suspension was sonicated for approximately 1 minute and delivered drop-wise to a background-corrected measuring cell, previously filled with toluene, until the obscuration reached the desired level. Volume distributions were obtained for three times. Upon measurement completion, the sample cell was emptied, cleaned, refilled with suspending medium and the sampling procedure repeated. For characterization, the values of D10, D50 and D90 (by volume) were specifically listed, each one being the mean of the six values available for each characterization parameter.

### Example 1

### Preparation of (±) 1-(isopropylamino)-3-[p-(2-methoxyethyl)phenoxy]-2-propanol succinate (2:1) (salt).

### A. Preparation of 1,2-epoxy-3-(4-(2-methoxyethyl)phenoxy)propane

To a 400 L reactor containing 49.6 kg of deionized water was added 7.93 kg (0.125 kmol) of potassium hydroxide pellets (88.25%) while maintaining the temperature below 30° C. The mixture was stirred until dissolution and then 20 kg (0.131 kmol) of 4-(2-methoxyethyl)phenol was added. The reactor was then closed, inertized, and the mixture was stirred for 20 minutes. During which time an opaline solution was obtained.

To the above mixture, 12.54 kg (0.135 kmol) of R,S-epichlorhydrin was added over 30 minutes. The reaction mixture, which had two layers, was then heated to 35±2° C. and kept at this temperature for 6±1 hours. Thereafter, 0.41 kg (0.0064 kmol) of potassium hydroxide pellets (88.25%) and 0.38 kg of deionized water were added. The reaction mixture was then maintained at 35±2° C. for 15±1 hours. Thereafter, the water-epichlorhydrin mixture was distilled under vacuum until 160 L were collected and an orange-colored liquid residue was obtained.

### B. Preparation of (±) 1-(isopropylamino)-3-[p-(2-methoxyethyl)phenoxy]-2-propanol

The distillation residue obtained in Step A above was next cooled to between 0-5° C., and 62.4 kg (1.056 kmol) of isopropylamine was added over 2-3 hours while maintaining the temperature below 15° C. to yield an orange-colored suspension. The reaction mixture was then heated to reflux temperature (50-55° C.) over 40 minutes, and maintained at reflux for 3 hours. The reaction mixture was then cooled to room temperature (20-25° C.), and the reaction evolution was monitored by HPLC. Next the reaction mixture was reheated, and excess isopropylamine was removed by distillation at atmospheric pressure while not exceeding 70±3° C.

The obtained residue was next cooled to 20-30° C., a vacuum was connected to the system, and the reactor was heated again to continue the distillation (under vacuum) until reaching 70±3° C. The reactor was then maintained at this temperature for 30-40 minutes, and an orange-colored oil was produced. Deionized water (7 kg) was then added to the obtained residue, the vacuum distillation was continued until reaching a temperature of 70±3° C., and the reactor was maintained at this temperature for 30-40 minutes.

Deionized water (30 kg) and toluene (38.2 kg) were then added to the resulting residue. The temperature of the mixture was then adjusted to room temperature (20-25° C.), and the mixture was stirred for 30 minutes. The layers were allowed to decant for 30 minutes and were then separated. The toluenic layer was washed first with 15 kg of deionized water, followed by a second washing with 5 kg of deionized water and 0.22 kg of hydrochloric acid 35% and was finally washed with 2×15 kg of deionized water. The resulting toluenic metoprolol base solution was weighed, and an aliquot was assayed for metoprolol base content.

### C. Preparation of Metoprolol Succinate Salt

The toluenic solution of metoprolol base obtained in Step B above was then charged to a previously inertized 400 L reactor. The solution was then distilled under vacuum, while ensuring that the internal temperature did not exceed 80° C., to yield an orange-colored, oily residue. The residue was then cooled to 60±5° C., and 49.4 kg of isopropanol was added. Next, the mixture was cooled to room temperature (20-25° C.), and the solution was transferred to a previously inertized 400 L reactor after passing through a 1 μm Cuno filter (3.2 kg of isopropanol were used for the washings).

Next, a solution of succinic acid in isopropanol was prepared by combining 0.221 kg (0.0019 kmol) of succinic acid per each kg (0.0037 kmol) of metoprolol base. The amount of metoprolol base was calculated from the data corresponding to the assay of the toluenic solution of metoprolol base. The succinic acid in isopropanol solution was then charged into the same reactor previously used for the isolation of metoprolol base, and the reactor was closed and inertized. Next, 98.8 kg of isopropanol was added, and the mixture was heated to 55-65° C. and maintained at this temperature for 15 minutes. While maintaining the temperature at 55-65° C., the succinic acid in isopropanol solution obtained was passed through a 1 μm Cuno filter and transferred to the reactor containing the previously prepared isopropanolic solution of metoprolol base. The solution of metoprolol base was also heated to 55-65° C. before receiving the succinic acid in isopropanol solution, and 3.2 kg of isopropanol was used for washings.

Once the addition was completed, the temperature was maintained at 55-65° C. for an additional 30 minutes, and then the mixture cooled to 20-25° C. over 2 hours and stirred for an additional 2 hours at that temperature. The suspension was then filtered and washed with 10 kg of filtered isopropanol.

The crude metoprolol succinate obtained was next charged to a 400 L reactor that was then closed and inertized. Next, 118 kg of filtered isopropanol was added, and the temperature was adjusted (if needed) to 20-25° C. The mixture was stirred at this temperature for 1 hour, and then filtered and washed with 10 kg of filtered isopropanol.

The wet product obtained was dried under vacuum for 6 hours at a temperature of 85-95° C. Particle size distribution at this stage of the production process was as follows: D (v, 0.1): below 6.5 μm, D (v, 0.5): below 24.7 μm, and D (v, 0.9): below 58.0 μm. The product was then milled and sieved (500 μm) to yield 28.8 kg (0.044 kmol) of metoprolol succinate (Yield: 67.15%).

Analytical data for metoprolol succinate: Loss on drying: less than 0.05%; sulphated ash: less than 0.05%; Assay (99.80%); XRD: substantially as shown in FIG. 1. Related impurities: max individual impurity less than 0.05%, total impurities 0.17%. Particle size distribution: D (v, 0.1): below 5.3 μm, D (v, 0.5): below 19.9 μm, and D (v, 0.9): below 53.7 μm. Tapped density: 0.53 g/ml. Isopropanol content: 386 ppm; Toluene content: 34 ppm.

### Example 2

### Preparation of (±) 1-(isopropylamino)-3-[p-(2-methoxyethyl)phenoxy]-2-propanol succinate (2:1) (salt).

This example was performed using the same quantities and conditions described in Example 1. The particle size distribution for this example is as follows:

Before milling: D (v, 0.1): below 6.9 μm, D (v, 0.5): below 22.8 μm, and D (v, 0.9): below 52.4 μm.

After milling: D (v, 0.1): below 4.9 μm, D (v, 0.5): below 18.0 μm, and D (v, 0.9): below 48.6 μm.

### Example 3

### Metoprolol Succinate Drying Process

Samples (1 g) of metoprolol succinate obtained according to the above process were dried for 24 hours at various temperatures (see Table 1, below). As illustrated in Table 1, the presence of degradation impurities was reported as % area HPLC.

TABLE 1

| | | % Area |
|---|---|---|
| Reference | Temperature | (RRT = 1.63-1.68) |
| 1 | 110° C. | 0.02 |
| 2 | 120° C. | 0.72 |
| 3 | 130° C. | 14.34 |

What is claimed is:

1. A process for preparing metoprolol and its pharmaceutically acceptable salts comprising:
i. reacting 4-(2-methoxyethyl)phenol with (R,S)-epichlorhydrin in an aqueous alkaline solution at approximately 35±2° C., wherein the aqueous alkaline solution is added in two portions;
ii. distilling under vacuum the resulting water-epichlorhydrin mixture to produce a first distillation residue;
iii. reacting said first distillation residue with isopropylamine to form a suspension while keeping the temperature below approximately 15° C.;
iv. heating said suspension thus obtained to reflux temperature and maintaining said suspension at reflux for approximately 3 hours;
v. removing excess isopropylamine by distillation at atmospheric pressure while ensuring that the reaction temperature does not exceed approximately 70±3° C.;
vi. extracting the resulting metoprolol base obtained with toluene as a toluenic solution of metoprolol base;
vii. distilling said toluenic solution of metoprolol base under vacuum while ensuring that the temperature does not exceed approximately 80° C.;
viii. cooling said distilled toluenic solution of metoprolol base to approximately 60±5° C., adding isopropanol, and cooling to room temperature;
ix. adding a solution of an acid in isopropanol to the isopropanolic solution of metoprolol base from the previous step, wherein said solution of acid in isopropanol has been heated to approximately 55° C. to approximately 65° C. and filtered prior to being added to said isopropanolic solution of metoprolol base;
x. cooling the resulting suspension of metoprolol salt to approximately 20° C. to approximately 25° C., stirring at this temperature for approximately 2 hours; and
xi. isolating a metoprolol salt.

2. The process of claim 1, wherein said acid added in step ix is succinic acid.

3. The process of claim 1, wherein said metoprolol salt is metoprolol succinate.

4. The process of claim 1, further comprising at least one of a milling step of said metoprolol salt and a sieving step said metoprolol salt.

5. The process of claim 1, further comprising drying said metoprolol salt at a temperature of approximately 85° C. to approximately 95° C.

6. The process of claim 1, further comprising drying said metoprolol salt at a temperature of approximately 90° C. to approximately 95° C.

7. The process of claim 1, wherein said metoprolol salt has a maximum loss on drying of approximately 0.2% of its weight.

8. Metoprolol succinate characterized by a powder X-ray spectrum substantially as shown in FIG. 1.

9. The metoprolol succinate of claim 8, wherein said metoprolol succinate is characterized by an X-Ray powder diffraction pattern (2Θ) (±0.2°) (XRD) having peaks at approximately 7.1, 11.5, 12.2, 13.1, 14.1, 14.4, 14.9, 17.2, 20.1, 21.2, 22.8, 23.1, 24.3, 24.6, 25.8, 26.2, 27.2, 30.1, 31.9, 33.4°.

10. A powder comprising metoprolol succinate, wherein said metoprolol succinate has a particle size distribution in which approximately 10% of the total volume comprises particles having a diameter below approximately 5 μm, approximately 50% of the total volume comprises particles having a diameter below about approximately 20 μm and approximately 90% of the total volume comprises particles having a diameter below approximately 55 μm.

11. The powder of claim 10, wherein said powder is prepared by a process that includes milling a metoprolol succinate feedstock having a mean particle size of approximately 25 μm.

12. A dosage unit comprising the powder of claim 10.

13. A dosage unit comprising metoprolol succinate, wherein the metoprolol succinate has a particle size distribution in which approximately 10% of the total volume comprises particles have a diameter below approximately 5 μm, approximately 50% of the total volume comprises particles have a diameter below about approximately 20 μm, and approximately 90% of the total volume comprises particles have a diameter below approximately 55 μm.
